(19) United States
(12) Patent Application Publication
(10) Pub. No.: US 2020/0231520 A1
(43) Pub. Date: Jul. 23, 2020

(54) ALKYLATION WITH OLEFIN MIXTURES

(71) Applicant: DuPont Industrial Biosciences USA, LLC, Wilmington, DE (US)

(72) Inventors: Diwakar Rana, Overland Park, KS (US); Jason Marshall Christian, Lenexa, KS (US)

(73) Assignee: DuPont Industrial Biosciences USA, LLC, Wilmington, DE (US)

(21) Appl. No.: 16/647,554

(22) PCT Filed: Sep. 14, 2018
(86) PCT No.: PCT/US2018/051075

§ 371 Date: Mar. 16, 2020

Related U.S. Application Data
(60) Provisional application No. 62/560,752, filed on Sep. 20, 2017.

(51) Int. Cl. C07C 2/62

(52) U.S. Cl. CPC C07C 2/62; C07C 9/16; C07C 2527/054

(57) ABSTRACT

This disclosure relates to alkylation processes. The process involves providing two or more reaction zones disposed in sequence. In at least the first two reaction zones, olefin mixture comprising C3 and C4 olefins is contacted with isoparaffin comprising isobutane in the presence of sulfuric acid solution under effective alkylation conditions to produce a product mixture comprising spent acid solution and alkylate product, wherein the molar ratio of C3 to C4 olefins in the olefin mixture decreases in each subsequent reaction zone. In the process, the sulfuric acid solution present in a reaction zone contains the spent acid solution produced in the immediately preceding reaction zone.

### CROSS-REFERENCE TO RELATED APPLICATION

This application claims priority of U.S. Patent Application 62/560,752 filed on Sep. 20, 2017, the disclosure of which is incorporated herein by reference in its entirety for all purposes.

### BACKGROUND

### Field of the Disclosure

The present disclosure relates to a sulfuric acid catalyzed alkylation process, and more specifically, to an alkylation process with olefin and acid staging.

### Description of Related Art

As a result of the curtailment in the use of tetraethyl lead as an octane-improving additive for gasoline, not only has the production of unleaded gasoline increased but the octane number specification of all grades of gasoline has increased as well.

In common oil refinery operations, gasoline products are created in several processing units. The collective volume of various kinds of the gasoline products created in oil refineries is referred to as the “gasoline pool” wherein alkylate is a popular blending component due to its low vapor pressure, low to zero olefins and aromatics contents, low sulfur content and high octane number.

Alkylation is a well-known refinery process for converting light olefins into high-octane gasoline components. Simply, alkylation involves the addition of an alkyl group to an organic molecule. Thus, an isoparaffin can be reacted with an olefin to produce isoparaffins of desired molecular weight known as alkylate. Generally, the alkylation of isoparaffins with olefins is accomplished by contacting the reactants with an acid acting catalyst such as hydrogen fluoride or sulfuric acid, settling the mixture to separate the catalyst from hydrocarbons, and further separating the hydrocarbons, usually by fractionation, to recover alkylate product. The resulting alkylate product is typically a mixture of C5 to C16 paraffinic isomers, with the exact composition depending upon the isoparaffin and olefin reactants utilized, as well as process conditions.

Historically, C4 olefins are preferred for use in alkylation, as they produce the highest octane alkylate with the lowest sulfuric acid catalyst consumption, on the order of about 0.2-0.4 pounds per gallon of alkylate product, as compared to propylene or C5 olefins. In fact, an acid consumption of 0.2-0.4 pounds per gallon of alkylate represents approximately ⅓ of the alkylation unit operating costs.

Alkylation feeds in which the olefins consist of 100% propylene or 100% amylenes typically result in much higher sulfuric acid catalyst consumption comparing with C4 olefins feed, representing considerably more than ⅓ of the alkylation operating cost. The higher sulfuric acid consumption, and lower octane numbers for the alkylate product, resulting from alkylation with propylenes and amylenes as compared to butylenes, can make these feed stocks economically unattractive, and therefore large concentrations of propylene and amylene are generally avoided in the alkylation feed.

### BRIEF SUMMARY OF THE DISCLOSURE

The present disclosure provides an alkylation process for producing alkylate. The process comprises: (a) providing three or more reaction zones disposed in sequence; (b) contacting a non-final olefin mixture comprising C3 and C4 olefins with an isoparaffin comprising isobutane in the presence of a non-final sulfuric acid solution under effective alkylation conditions in each non-final reaction zone to produce a product mixture comprising a non-final spent acid solution and an alkylate product; and (c) contacting a final olefin comprising an olefin selected from the group consisting of C4 olefins, C5 olefins and mixtures thereof with an isoparaffin comprising isobutane in the presence of a final sulfuric acid solution under effective alkylation conditions in a final reaction zone to produce a product mixture comprising a final spent acid solution and an alkylate product; wherein the molar ratio of C3 to C4 olefins in the non-final olefin mixture decreases in each subsequent non-final reaction zone, and the sulfuric acid solution present in a reaction zone comprises the spent acid solution produced in the immediately preceding reaction zone.

The present disclosure provides another alkylation process for producing alkylate. The process comprises: (a) providing three reaction zones disposed in sequence; (b) contacting a first olefin mixture comprising C3 and C4 olefins with an isoparaffin comprising isobutane in the presence of a first sulfuric acid solution under effective alkylation conditions in a first reaction zone to produce a product mixture comprising a first spent acid solution and an alkylate product; (c) contacting a second olefin mixture comprising C3 and C4 olefins with an isoparaffin comprising isobutane in the presence of a second sulfuric acid solution comprising the first spent acid solution under effective alkylation conditions in a second reaction zone to produce a product mixture comprising a second spent acid solution and an alkylate product; and (d) contacting a final olefin comprising an olefin selected from the group consisting of C4 olefins, C5 olefins and mixtures thereof with an isoparaffin comprising isobutane in the presence of a final sulfuric acid solution comprising the second spent acid solution under effective alkylation conditions in a final reaction zone to produce a product mixture comprising a final spent acid solution and an alkylate product; wherein the molar ratio of C3 to C4 olefins in the first olefin mixture is in a range of from about 99:1 to about 60:40, the molar ratio of C3 to C4 olefins in the second olefin mixture is in a range of from about 70:30 to about 30:70, and the molar ratio of C3 to C4 olefins in the first olefin mixture is higher than the molar ratio of C3 to C4 olefins in the second olefin mixture.

The present disclosure provides another alkylation process for producing alkylate. The process comprises: (a) providing four reaction zones disposed in sequence; (b) contacting a first olefin mixture comprising C3 and C4 olefins with an isoparaffin comprising isobutane in the presence of a first sulfuric acid solution under effective alkylation conditions in a first reaction zone to produce a product mixture comprising a first spent acid solution and an alkylate product; (c) contacting a second olefin mixture comprising C3 and C4 olefins with an isoparaffin comprising isobutane in the presence of a second sulfuric acid solution comprising the first spent acid solution under effective alkylation conditions in a second reaction zone to produce a product mixture comprising a second spent acid solution and an alkylate product; (d) contacting a third olefin comprising a C4 olefin with an isoparaffin comprising isobutane in the presence of a third sulfuric acid solution comprising the second spent acid solution under effective alkylation conditions in a third reaction zone to produce a product mixture comprising a third spent acid solution and an alkylate product; and (e) contacting a final olefin comprising a C5 olefin with an isoparaffin comprising isobutane in the presence of a final sulfuric acid solution comprising the third spent acid solution under effective alkylation conditions in a final reaction zone to produce a product mixture comprising a final spent acid solution and an alkylate product; wherein the molar ratio of C3 to C4 olefins in the first olefin mixture is in a range of from about 99:1 to about 60:40, the molar ratio of C3 to C4 olefins in the second olefin mixture is in a range of from about 70:30 to about 30:70, and the molar ratio of C3 to C4 olefins in the first olefin mixture is higher than the molar ratio of C3 to C4 olefins in the second olefin mixture.

The present disclosure provides another alkylation process for producing alkylate. The process comprises: (a) providing two reaction zones disposed in sequence; (b) contacting a first olefin mixture comprising C3 and C4 olefins with an isoparaffin comprising isobutane in the presence of a first sulfuric acid solution under effective alkylation conditions in a first reaction zone to produce a product mixture comprising a first spent acid solution and an alkylate product; and (c) contacting a second olefin mixture comprising C3 and C4 olefins with an isoparaffin comprising isobutane in the presence of a second sulfuric acid solution comprising the first spent acid solution under effective alkylation conditions in a second reaction zone to produce a product mixture comprising a second spent acid solution and an alkylate product; wherein the molar ratio of C3 olefin to C4 olefins in the first olefin mixture is in a range of from about 99:1 to about 60:40, the molar ratio of C3 olefin to C4 olefins in the second olefin mixture is in a range of from about 70:30 to about 30:70, and the molar ratio of C3 olefin to C4 olefins in the first olefin mixture is higher than the molar ratio of C3 olefin to C4 olefins in the second olefin mixture.

### DETAILED DESCRIPTION

The foregoing general description and the following detailed description are exemplary and explanatory only and are not restrictive of the invention, as defined in the appended claims. Other features and benefits of any one or more of the embodiments will be apparent from the following detailed description, and from the claims.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or”. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

When an amount, concentration, or other value or parameter is given as a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range.

Before addressing details of embodiments described below, some terms are defined or clarified.

The term “reaction zones disposed in sequence”, as used herein, means that the non-final spent acid solution produced in each non-final reaction zone is sent to the immediately subsequent reaction zone as part or all of the sulfuric acid solution therein, that is, non-final spent acid solution from a non-final reaction zone can be reused as catalyst in the immediately subsequent reaction zone. In some embodiments, a portion of a non-final spent acid solution produced in a non-final reaction zone is recycled to the same non-final reaction zone, and the portion of the non-final spent acid solution not recycled is sent to the immediately subsequent reaction zone as part or all of the sulfuric acid solution therein. In some embodiments, a portion of a final spent acid solution produced in the final reaction zone is recycled to the final reaction zone, and the rest (the portion of the final spent acid solution not recycled) is purged, that is, the final spent acid solution is not reused in a non-final reaction zone.

The term “spent acid solution”, as used herein, means the sulfuric acid solution exiting a reaction zone at the end of the alkylation reaction. Typically, a spent acid solution comprises sulfuric acid, water, acid soluble oils, and reaction intermediates such as sulfate esters. In some embodiments, the spent acid solution exits a reaction zone in the form of a sulfuric acid/hydrocarbon emulsion. The sulfuric acid/hydrocarbon emulsion can be introduced into a sulfuric acid settler wherein a hydrocarbon phase is allowed to separate from a sulfuric acid phase of the sulfuric acid/hydrocarbon emulsion. A portion of the sulfuric acid phase comprising, consisting essentially of, or consisting of the spent acid solution from a reaction zone can be recycled to the same reaction zone. Another portion or the non-recycled portion of the sulfuric acid phase comprising, consisting essentially of, or consisting of the spent acid solution from the reaction zone can be directed to or introduced into the immediately subsequent reaction zone as part or all of the sulfuric acid solution therein.

The term “olefin”, as used herein, means an unsaturated hydrocarbon comprising one or more carbon-carbon double bonds. The unsaturated hydrocarbon herein does not include aromatic compounds. In some embodiments, the olefin has a single carbon-carbon double bond. In some embodiments, the olefin comprises no more than about 10 mole %, 5 mole %, 3 mole %, 2 mole %, 1 mole %, 0.5 mole %, 0.2 mole %, or 0.1 mole % of ethylene and olefins having six or more carbons based on the total amount of olefins. In some embodiments, the olefin is essentially free of ethylene and olefins having six or more carbons. In some embodiments, the olefin comprises no more than about 10 mole %, 5 mole %, 3 mole %, 2 mole %, 1 mole %, 0.5 mole %, 0.2 mole %, or 0.1 mole % of dienes such as butadiene and propadiene based on the total amount of olefins. In some embodiments, the olefin is essentially free of dienes.

The term “alkylate”, as used herein, means the reaction products generated in alkylation reactions between an olefin and an isoparaffin in the presence of a sulfuric acid catalyst. Alkylates typically are highly branched paraffinic hydrocarbons. Refiners use alkylate as a gasoline blend stock to boost octane, reduce Reid vapor pressure (“RVP”), and reduce olefin content in the final gasoline blend.

The term “acid strength”, as used herein, means the concentration of the sulfuric acid solution which is expressed in weight percent H₂SO₄ as determined by titration with standardized sodium hydroxide. Diluents that can reduce the acid strength of a sulfuric acid solution include water, acid soluble oils formed by side reactions during an alkylation process, and reaction intermediates such as sulfate esters formed during an alkylation reaction.

The term “acid consumption”, as used herein, means the amount of sulfuric acid solution “consumed” during an alkylation process in comparison with the amount of alkylate generated therein through alkylation reactions between olefin and isoparaffin. During an alkylation process, the sulfuric acid solution is diluted by water contained in the hydrocarbon feeds and acid soluble oils formed by side reactions. Additionally, formation of stable intermediates such as sulfate esters also dilute the sulfuric acid solution. The sulfuric acid solution is generally spent at about 80 to 94 wt % H₂SO₄, depending on the kind of olefin involved in the reaction, in order to avoid an acid runaway reaction. In this disclosure, acid consumption in an alkylation process is calculated by carrying out the alkylation reaction with a fresh sulfuric acid solution starting material having acid strength of about 99.2 wt % and dividing (i) the total amount of the fresh sulfuric acid solution fed to the reaction zone(s) by (ii) the total amount of alkylate generated through alkylation reactions between olefin and isoparaffin during the process. Typically, the fresh sulfuric acid solution is fed to the first reaction zone and optionally can also be fed to one or more subsequent reaction zone(s).

The term “fresh sulfuric acid solution”, as used herein, means a sulfuric acid solution which has not been used as the catalyst in an alkylation reaction.

The fresh sulfuric acid solution is essentially free of acid soluble oils and alkylation intermediates such as sulfate esters.

The term “olefin space velocity”, as used herein, means the volume of olefins fed to the reaction zone per hour divided by the average volume of the sulfuric acid solution in the reaction zone.

The term “wt %”, as used herein, means percentage by weight.

The term “mole %”, as used herein, means percentage by molar amount.

Alkylation processes in general are well known to those of skill in the art. For example, see “Catalytic Alkylation”, Petri/Chem Engineer, December 1961 and January 1962, “Alkylation will be key process in reformulated gasoline era”, Oil & Gas Journal, Nov. 12, 1990, pp. 79-92, “H₂SO₄, HF processes compared, and new technologies revealed”, Oil & Gas Journal, Nov. 26, 1990, pp. 70-77, and “Which alkylation—HF or H₂SO₄?”, Hydrocarbon Processing, September 1985, all herein incorporated by reference in its entirety for all purposes. Additionally, alkylation is generally disclosed in U.S. Pat. Nos. 4,018,846; 4,225,740; 4,276,731; 4,371,731; 4,383, 977; 4,404,418; 4,467,131; 4,513,165; 4,777,323; and 5,157,196; all herein also incorporated by reference in its entirety for all purposes.

Although propylene and amylenes are less favorable than butylenes in an alkylation process, many refiners have mixed olefins containing large amounts of propylene and/or amylenes which must be alkylated. Additionally, due to the low cost of propylene or amylene feed stocks, the low value of propylene as a petrochemical feed stock, the low cost of isobutane feed stocks, reformulated gasoline requirements, Reid Vapor Pressure (“RVP”), D-86 distillation temperatures, and other requirements, increasing amounts of propylenes and amylenes are being alkylated and added to the gasoline pool.

Thus, while C4 olefins are preferred for use in alkylation because of their low acid consumption, and resulting high-octane alkylate, there is a need for an improved alkylation with propylene. Other reasons for alkylating with propylene and/or amylenes include their ready availability, and unavailability of sufficient amounts of C4 olefins for the amount of alkylate desired. Removing amylenes from the gasoline blending pool is another reason for alkylating with amylene.

The present disclosure provides an alkylation process for producing alkylate. The process comprises: (a) providing three or more reaction zones disposed in sequence; (b) contacting a non-final olefin mixture comprising C3 and C4 olefins with an isoparaffin comprising isobutane in the presence of a non-final sulfuric acid solution under effective alkylation conditions in each non-final reaction zone to produce a product mixture comprising a non-final spent acid solution and an alkylate product; and (c) contacting a final olefin comprising an olefin selected from the group consisting of C4 olefins, C5 olefins and mixtures thereof with an isoparaffin comprising isobutane in the presence of a final sulfuric acid solution under effective alkylation conditions in a final reaction zone to produce a product mixture comprising a final spent acid solution and an alkylate product; wherein the molar ratio of C3 to C4 olefins in the non-final olefin mixture decreases in each subsequent non-final reaction zone, and the sulfuric acid solution present in a reaction zone comprises the spent acid solution produced in the immediately preceding reaction zone.

In this disclosure, each reaction zone can independently comprise one or more alkylation reactors. In one embodiment, three alkylation reaction zones are provided and disposed in sequence. In another embodiment, four alkylation reaction zones are provided and disposed in sequence. In another embodiment, two alkylation reaction zones are provided and disposed in sequence.

During an alkylation process, an olefin is reacted with an isoparaffin in the presence of a sulfuric acid catalyst to form an alkylate. In some embodiments of this disclosure, the olefin feed to a non-final reaction zone (i.e., a reaction zone preceding the final reaction zone) is a non-final olefin mixture comprising C3 and C4 olefins.

In some embodiments, the non-final olefin mixture comprising C3 and C4 olefins is essentially free of C5 olefins. In some embodiments, the non-final olefin mixture comprising C3 and C4 olefins comprises no more than about 30 mole %, 20 mole %, 10 mole %, 5 mole %, 3 mole %, 2 mole %, 1 mole %, 0.5 mole %, 0.2 mole %, or 0.1 mole % of C5 olefins based on the total amount of olefins. In some embodiments, the non-final olefin mixture comprising C3 and C4 olefins consists essentially of, or consists of C3 and C4 olefins.

C3 olefin is propene. Olefins with four or more carbons have isomers. In this disclosure, a C4 olefin (i.e., butene, also known as butylene) is selected from the group consisting of 1-butene, 2-butene, isobutylene (i.e., 2-methylpropene), and combinations thereof. 2-butene includes cis-2-butene and trans-2-butene. A C5 olefin (i.e., pentene, also known as amylene) is selected from the group consisting of 1-pentene and its branched isomers, 2-pentene and its branched isomers, and combinations thereof. 2-Pentene includes cis-2-pentene and trans-2-pentene.

In some embodiments, the C4 olefin comprises at least about 90 mole %, 95 mole %, or 98 mole % of isobutylene based on the total amount of C4 olefin. In some embodiments, the C4 olefin comprises from about 25 mole % to about 30 mole % of isobutylene based on the total amount of C4 olefin. In some embodiments, the C4 olefin comprises no more than about 8 mole %, 5 mole %, 2 mole %, 1 mole %, 0.5 mole %, or 0.2 mole % of isobutylene based on the total amount of C4 olefin.

In some embodiments of this disclosure, the molar ratio of C3 to C4 olefins in the non-final olefin mixture comprising C3 and C4 olefins decreases in each subsequent reaction zone preceding the final reaction zone, that is, from the first reaction zone to the reaction zone immediately preceding the final reaction zone, the molar ratio of C3 olefin to C4 olefins in the olefin mixture fed to each subsequent reaction zone decreases. In some embodiments, the molar ratio of C3 to C4 olefins in an olefin mixture fed to a reaction zone decreases in each subsequent reaction zone, that is, if the final olefin also comprises C3 and C4 olefins, the molar ratio of C3 to C4 olefins in the final olefin is smaller than the molar ratio of C3 to C4 olefins in the olefin mixture fed to the reaction zone immediately preceding the final reaction zone. It was found through experiments that the acid consumption during the process can be reduced with such olefin staging design.

In some embodiments, the alkylation process has acid consumption of no more than about 0.7 lb/gal (pound per gallon). In some embodiments, acid consumption in the alkylation process is no more than about 0.6 lb/gal. In some embodiments, acid consumption is no more than about 0.5 lb/gal. In some embodiments, acid consumption is no more than about 0.4 lb/gal.

In some embodiments, the molar ratio of C3 to C4 olefins in the non-final olefin mixture comprising C3 and C4 olefins is in a range of from about 99:1 to about 20:80. In some embodiments, the molar ratio of C3 to C4 olefins in the non-final olefin mixture comprising C3 and C4 olefins is in a range of from about 95:5 to about 30:70. In some embodiments, the molar ratio of C3 to C4 olefins in the non-final olefin mixture comprising C3 and C4 olefins is in a range of from about 90:10 to about 30:70. In some embodiments, the molar ratio of C3 to C4 olefins in the non-final olefin mixture comprising C3 and C4 olefins is in a range of from about 90:10 to about 40:60.

An isoparaffin in this disclosure means an isoparaffin reactant for the alkylation reaction. In some embodiments, the isoparaffin comprises at least about 80 mole % of isobutane based on the total amount of the isoparaffin. In some embodiments, the isoparaffin comprises at least about 85 mole %, 90 mole %, 95 mole %, or 98 mole % of isobutane based on the total amount of the isoparaffin. In some embodiments, the isoparaffin consists essentially of, or consists of isobutane.

In some embodiments, the isoparaffin comprises, consists essentially of, or consists of isobutane and isopentane. In some embodiments, the isopentane content in the isoparaffin is no more than about 30 mole %, 20 mole %, 10 mole %, 5 mole %, 2 mole %, 1 mole %, 0.5 mole %, 0.2 mole %, or 0.1 mole % based on the total amount of the isoparaffin. In some embodiments, the isoparaffin is essentially free of isopentane.

Typically, the isoparaffin comprises no more than about 10 mole %, 5 mole %, 2 mole %, 1 mole %, 0.5 mole %, 0.2 mole %, or 0.1 mole % of isoparaffins having six or more carbons based on the total amount of the isoparaffin. In some embodiments, the isoparaffin is essentially free of isoparaffins having six or more carbons.

Olefin and isoparaffin can be fed to the reaction zone using well-known chemical engineering practice. In some embodiments, olefin and isoparaffin are fed to the reaction zone separately. In some embodiments, olefin and isoparaffin are combined or admixed upstream of the reaction zone. Typically, alkylation reactions of this disclosure are carried out with molar ratio of isoparaffin to olefin fed to the reaction zone of greater than 1 to minimize undesired polymerization reactions. The term “molar ratio of isoparaffin to olefin fed to the reaction zone”, as used herein, means the molar ratio of the total amount of isoparaffin to the total amount of olefin fed to the reaction zone. The total amount of isoparaffin fed to the reaction zone includes freshly fed isoparaffin and recycled isoparaffin (if present). The total amount of olefin fed to the reaction zone includes freshly fed olefin and recycled olefin (if present). Typically, the molar ratio of isoparaffin to olefin fed to each reaction zone is in a range of from about 2:1 to about 50:1, and in some embodiments is in a range of from about 4:1 to about 20:1. In some embodiments, the molar ratio is in a range of from about 5:1 to about 12:1.

Typically, olefin and isoparaffin are only a part of the total hydrocarbons fed to an alkylation reaction zone. In this disclosure, olefin or olefin mixture means the olefin component(s) in the hydrocarbon feeds to the reaction zone, and isoparaffin means the isoparaffin component(s) in the hydrocarbon feeds to the reaction zone. Hydrocarbon feeds to an alkylation reaction zone can also comprise diluents which are inert compounds under the effective alkylation conditions of this disclosure. In some embodiments, a diluent comprises, consists essentially of, or consists of a straight chain paraffin (i.e., n-paraffin). In some embodiments, a diluent comprises, consists essentially of, or consists of n-paraffin selected from the group consisting of propane, n-butane, n-pentane and combinations thereof. In some embodiments, a diluent comprises, consists essentially of, or consists of propane and n-butane.

The term “diluent ratio” refers to the molar ratio of diluent to isoparaffin in the hydrocarbon feeds to an alkylation reaction zone. In some embodiments, a diluent is also fed to one or more reaction zones as part of the hydrocarbon feeds in the alkylation process. In some embodiments, a diluent is also fed to each reaction zone in the alkylation process. In some embodiments, the diluent ratio is no more than about 0.8, 0.7, 0.6, 0.5, 0.4, 0.3, 0.2, or 0.1.

In alkylation process, a sulfuric acid solution is used as a catalyst for alkylation reactions. In this disclosure, the sulfuric acid solution is an aqueous solution comprising sulfuric acid. Typically, in a reaction zone, the volume ratio of the sulfuric acid solution to hydrocarbon is in a range of from about 0.5:1 to about 2.5:1. In some embodiments, the volume ratio of the sulfuric acid solution to hydrocarbon in a reaction zone is in a range of from about 0.7:1 to about 2.3:1, or from about 0.8:1 to about 2.0:1, or from about 0.9:1 to about 1.8:1, or from about 1:1 to about 1.5:1, or from about 1:1 to about 1.2:1.

The alkylation reactions can be carried out under effective alkylation conditions in a reaction zone to generate an alkylate product. For example, the temperature in the reaction zone can be in a range of from about 0° C. to about 30° C. In some embodiments, the temperature in the reaction zone is in a range of from about 4° C. to about 20° C., or from about 6° C. to about 16° C., or from about 7° C. to about 12° C. The pressure in the reaction zone can be in a range of from about 1 to about 100 psig, or from about 30 to about 80 psig, or from about 40 to about 70 psig. In some embodiments, the olefin space velocity is in a range of from about 0.2 to about 0.7 per hour. In some embodiments, the olefin space velocity is in a range of from about 0.3 to about 0.6 per hour, or from about 0.3 to about 0.5 per hour, or from about 0.3 to about 0.4 per hour, or from about 0.4 to about 0.5 per hour, or from about 0.6 to about 0.7 per hour.

In this disclosure, the product mixture or effluent from a reaction zone comprises a spent acid solution and an alkylate product. Such spent acid solution from a reaction zone preceding the final reaction zone can be directed to the immediately subsequent reaction zone as part or all of the sulfuric acid solution therein, that is, the sulfuric acid solution present in a reaction zone subsequent to the first reaction zone comprises, consists essentially of, or consists of the spent acid solution produced in the immediately preceding reaction zone.

Typically, the product mixture or effluent from a reaction zone is an emulsion comprising alkylate, sulfuric acid, and unreacted isoparaffin. The product mixture or effluent can be separated into a sulfuric acid phase and a hydrocarbon phase, and the sulfuric acid phase comprising spent acid solution can be recycled to the reaction zone and/or transferred to the immediately subsequent reaction zone as a source of sulfuric acid. In some embodiments, a portion of the sulfuric acid phase comprising spent acid solution is recycled to the same reaction zone that produced such spent acid solution, and the non-recycled portion of the sulfuric acid phase is introduced into the immediately subsequent reaction zone.

The sulfuric acid catalyst is least effective at low acid strengths. In some embodiments, the sulfuric acid solution in each reaction zone has acid strength in a range of from about 80 to about 99 percent. In some embodiments, the sulfuric acid solution in each reaction zone has acid strength in a range of from about 88 to about 97 percent. In some embodiments, the acid strength of the sulfuric acid solution decreases in each subsequent reaction zone.

A fresh sulfuric acid solution used for the alkylation process is typically an aqueous solution of sulfuric acid having acid strength in a range of from about 96.5% to about 99.5%. In some embodiments, the first sulfuric acid solution is a fresh sulfuric acid solution, that is, in the first reaction zone, the first olefin mixture comprising C3 and C4 olefins is contacted with an isoparaffin comprising isobutane in the presence of a fresh sulfuric acid solution. The acid strength decreases as the alkylation process carries on and the sulfuric acid solution is diluted as a result of the side reactions and feed contaminants to form a spent acid solution. In some embodiments, an additional sulfuric acid solution having higher acid strength than the spent acid solution can be added to the spent acid solution to restore some or substantially all of the acid strength. It was found through experiments that although more acids were used for the alkylation process, the additional sulfuric acid can increase the alkylate production and as a result, reduce the acid consumption of the process.

In some embodiments, the sulfuric acid solution present in a second reaction zone further comprises an additional sulfuric acid solution having higher acid strength than the spent acid solution produced in a first reaction zone. In some embodiments, the sulfuric acid solution present in a reaction zone between the first reaction zone and the final reaction zone (i.e., a reaction zone subsequent to a first reaction zone but preceding the final reaction zone) further comprises an additional sulfuric acid solution having higher acid strength than the spent acid solution produced in the immediately preceding reaction zone. In some embodiments, the sulfuric acid solution present in each reaction zone between the first reaction zone and the final reaction zone further comprises an additional sulfuric acid solution having higher acid strength than the spent acid solution produced in the immediately preceding reaction zone. A person of ordinary skill in the art appreciates that in these embodiments, the sulfuric acid solution present in a reaction zone is a homogeneous mixture of the spent acid solution produced in the immediately preceding reaction zone and the newly added additional sulfuric acid solution.

In some embodiments, the additional sulfuric acid solution is a fresh sulfuric acid solution. In some embodiments, the additional sulfuric acid solution has acid strength in a range of from about 96.5 percent to about 99.5 percent. In some embodiments, the additional sulfuric acid solution is admixed with the spent acid solution produced in the immediately preceding reaction zone before being fed to the reaction zone. In some embodiments, sufficient amount of the additional sulfuric acid solution is added to the reaction zone to maintain the acid strength of the sulfuric acid solution in a range of from about 80 to about 99 percent or from about 88 to about 97 percent.

In some embodiments of this disclosure, in the final reaction zone, a final olefin comprising an olefin selected from the group consisting of C4 olefins, C5 olefins and mixtures thereof is contacted with an isoparaffin comprising isobutane in the presence of a final sulfuric acid solution under effective alkylation conditions to produce a final spent acid solution and an alkylate product.

In some embodiments, the final olefin is essentially free of C3 olefin (i.e., propene, also known as propylene). In some embodiments, the final olefin comprises no more than about 50 mole %, 40 mole %, 30 mole %, 20 mole %, 10 mole %, 7 mole %, 5 mole %, 3 mole %, 2 mole %, 1 mole %, 0.5 mole %, 0.2 mole %, or 0.1 mole % of C3 olefin based on the total amount of the final olefin. In some embodiments, the final olefin consists essentially of, or consists of C4 and C5 olefins. In some embodiments, the final olefin consists essentially of, or consists of C4 olefins. In some embodiments, the final olefin consists essentially of, or consists of C5 olefins.

In some embodiments, the final olefin comprises C4 olefin(s) essentially free of C3 and C5 olefins. In some embodiments, the final olefin comprises C5 olefin(s) essentially free of C3 and C4 olefins.

In some embodiments, the final sulfuric acid solution in the final reaction zone is essentially free of additional sulfuric acid solution, that is, essentially no additional sulfuric acid solution is fed to the final reaction zone other than the spent acid solution produced in the immediately preceding reaction zone. In some embodiments, the final sulfuric acid solution in the final reaction zone consists essentially of, or consists of the spent acid solution produced in the immediately preceding reaction zone.

In some embodiments, the final spent acid solution has acid strength in a range of from about 80 percent to about 94 percent. In some embodiments, the final spent acid solution has acid strength in a range of from about 82 percent to about 92 percent. In some embodiments, the final spent acid solution has acid strength in a range of from about 83 percent to about 91 percent, or from about 83 percent to about 87 percent, or from about 83 percent to about 85 percent, or from about 80 percent to about 84 percent, or from about 80 percent to about 82 percent, or from about 85 percent to about 90 percent, or from about 89 percent to about 91 percent.

In some embodiments, the final olefin comprises C4 olefin(s) essentially free of C3 and C5 olefins, and the final spent acid solution has acid strength in a range of from about 87 percent to about 91 percent, or from about 87 percent to about 89 percent. In some embodiments, the final olefin comprises C5 olefin(s) essentially free of C3 and C4 olefins, and the final spent acid solution has acid strength in a range of from about 80 percent to about 85 percent, or from about 80 percent to about 82 percent.

In some embodiments, the alkylation process is conducted in three reaction zones disposed in sequence. In such embodiments, the alkylation process comprises: (a) providing three reaction zones disposed in sequence; (b) contacting a first olefin mixture comprising C3 and C4 olefins with an isoparaffin comprising isobutane in the presence of a first sulfuric acid solution under effective alkylation conditions in a first reaction zone to produce a product mixture comprising a first spent acid solution and an alkylate product; (c) contacting a second olefin mixture comprising C3 and C4 olefins with an isoparaffin comprising isobutane in the presence of a second sulfuric acid solution comprising the first spent acid solution under effective alkylation conditions in a second reaction zone to produce a product mixture comprising a second spent acid solution and an alkylate product; and (d) contacting a final olefin comprising an olefin selected from the group consisting of C4 olefins, C5 olefins and mixtures thereof with an isoparaffin comprising isobutane in the presence of a final sulfuric acid solution comprising the second spent acid solution under effective alkylation conditions in a final reaction zone to produce a product mixture comprising a final spent acid solution and an alkylate product; wherein the molar ratio of C3 olefin to C4 olefins in the first olefin mixture is in a range of from about 99:1 to about 60:40, the molar ratio of C3 olefin to C4 olefins in the second olefin mixture is in a range of from about 70:30 to about 30:70, and the molar ratio of C3 olefin to C4 olefins in the first olefin mixture is higher than the molar ratio of C3 olefin to C4 olefins in the second olefin mixture. In some embodiments, the final sulfuric acid solution in the final reaction zone consists essentially of, or consists of the second spent acid solution, that is, the final sulfuric acid solution is essentially free of additional sulfuric acid solution.

The present disclosure also provides an alkylation process for producing alkylate. The process comprises: (a) providing four reaction zones disposed in sequence; (b) contacting a first olefin mixture comprising C3 and C4 olefins with an isoparaffin comprising isobutane in the presence of a first sulfuric acid solution under effective alkylation conditions in a first reaction zone to produce a product mixture comprising a first spent acid solution and an alkylate product; (c) contacting a second olefin mixture comprising C3 and C4 olefins with an isoparaffin comprising isobutane in the presence of a second sulfuric acid solution comprising the first spent acid solution under effective alkylation conditions in a second reaction zone to produce a product mixture comprising a second spent acid solution and an alkylate product; (d) contacting a third olefin comprising a C4 olefin with an isoparaffin comprising isobutane in the presence of a third sulfuric acid solution comprising the second spent acid solution under effective alkylation conditions in a third reaction zone to produce a product mixture comprising a third spent acid solution and an alkylate product; and (e) contacting a final olefin comprising a C5 olefin with an isoparaffin comprising isobutane in the presence of a final sulfuric acid solution comprising the third spent acid solution under effective alkylation conditions in a final reaction zone to produce a product mixture comprising a final spent acid solution and an alkylate product; wherein the molar ratio of C3 to C4 olefins in the first olefin mixture is in a range of from about 99:1 to about 60:40, the molar ratio of C3 to C4 olefins in the second olefin mixture is in a range of from about 70:30 to about 30:70, and the molar ratio of C3 to C4 olefins in the first olefin mixture is higher than the molar ratio of C3 to C4 olefins in the second olefin mixture. In some embodiments, the final spent acid solution has acid strength in a range of from about 80 percent to about 86 percent, or from about 80 percent to about 84 percent.

In some embodiments, the third olefin comprising a C4 olefin is essentially free of C3 olefin. In some embodiments, the third olefin comprises no more than about 50 mole %, 40 mole %, 30 mole %, 20 mole %, 10 mole %, 7 mole %, 5 mole %, 3 mole %, 2 mole %, 1 mole %, 0.5 mole %, 0.2 mole %, or 0.1 mole % of C3 olefin based on the total amount of the third olefin. In some embodiments, the third olefin comprising a C4 olefin is essentially free of C5 olefin. In some embodiments, the third olefin comprises no more than about 30 mole %, 20 mole %, 10 mole %, 7 mole %, 5 mole %, 3 mole %, 2 mole %, 1 mole %, 0.5 mole %, 0.2 mole %, or 0.1 mole % of C5 olefin based on the total amount of the third olefin. In some embodiments, the third olefin consists essentially of, or consists of a C4 olefin.

In some embodiments, the final olefin comprising a C5 olefin is essentially free of C3 olefin. In some embodiments, the final olefin comprises no more than about 30 mole %, 20 mole %, 10 mole %, 7 mole %, 5 mole %, 3 mole %, 2 mole %, 1 mole %, 0.5 mole %, 0.2 mole %, or 0.1 mole % of C3 olefin based on the total amount of the final olefin. In some embodiments, the final olefin comprising a C5 olefin is essentially free of C4 olefin. In some embodiments, the final olefin comprises no more than about 30 mole %, 20 mole %, 10 mole %, 7 mole %, 5 mole %, 3 mole %, 2 mole %, 1 mole %, 0.5 mole %, 0.2 mole %, or 0.1 mole % of C4 olefin based on the total amount of the final olefin. In some embodiments, the final olefin consists essentially of, or consists of a C5 olefin.

In some embodiments, the third sulfuric acid solution in the third reaction zone is essentially free of additional sulfuric acid solution, that is, essentially no additional sulfuric acid solution is fed to the third reaction zone other than the second spent acid solution. In some embodiments, the third sulfuric acid solution consists essentially of, or consists of the second spent acid solution. In some embodiments, the third spent acid solution has acid strength in a range of from about 87 percent to about 93 percent.

The present disclosure also provides an alkylation process for producing alkylate. The process comprises: (a) providing two reaction zones disposed in sequence; (b) contacting a first olefin mixture comprising C3 and C4 olefins with an isoparaffin comprising isobutane in the presence of a first sulfuric acid solution under effective alkylation conditions in a first reaction zone to produce a product mixture comprising a first spent acid solution and an alkylate product; and (c) contacting a second olefin mixture comprising C3 and C4 olefins with an isoparaffin comprising isobutane in the presence of a second sulfuric acid solution comprising the first spent acid solution under effective alkylation conditions in a second reaction zone to produce a product mixture comprising a second spent acid solution and an alkylate product; wherein the molar ratio of C3 olefin to C4 olefins in the first olefin mixture is in a range of from about 99:1 to about 60:40, the molar ratio of C3 olefin to C4 olefins in the second olefin mixture is in a range of from about 70:30 to about 30:70, and the molar ratio of C3 olefin to C4 olefins in the first olefin mixture is higher than the molar ratio of C3 olefin to C4 olefins in the second olefin mixture. In such embodiments, the second reaction zone is the final reaction zone, and the second spent acid solution is the final spent acid solution, that is, no reaction zone is subsequent to the second reaction zone. In some embodiments, the second sulfuric acid solution in the second reaction zone consists essentially of, or consists of the first spent acid solution, that is, the second sulfuric acid solution is essentially free of additional sulfuric acid solution. In some embodiments, the second sulfuric acid solution has an acid strength in a range of from about 90 to about 96 percent. In some embodiments, the second sulfuric acid solution has an acid strength in a range of from about 90 to about 94 percent.

Typically, the molar ratio of C3 olefin to C4 olefins in the first olefin mixture is in a range of from about 99:1 to about 60:40. In some embodiments, the molar ratio of C3 olefin to C4 olefins in the first olefin mixture is in a range of from about 95:5 to about 65:35, or from about 90:10 to about 70:30, or from about 90:10 to about 75:25, or from about 90:10 to about 80:20.

Typically, the molar ratio of C3 olefin to C4 olefins in the second olefin mixture is in a range of from about 80:20 to about 20:80. In some embodiments, the molar ratio of C3 olefin to C4 olefins in the second olefin mixture is in a range of from about 75:25 to about 25:75, or from about 70:30 to about 30:70, or from about 65:35 to about 35:65, or from about 60:40 to about 40:60, or from about 55:45 to about 45:55. In some embodiments, the molar ratio of C3 olefin to C4 olefins in the second olefin mixture is about 50:50.

In some embodiments, the first olefin mixture and/or the second olefin mixture is essentially free of C5 olefins. In some embodiments, the first olefin mixture and/or the second olefin mixture comprises no more than about 10 mole %, 5 mole %, 3 mole %, 2 mole %, 1 mole %, 0.5 mole %, 0.2 mole %, or 0.1 mole % of C5 olefins based on the total amount of the olefin mixture. In some embodiments, the first olefin mixture and/or the second olefin mixture consists essentially of, or consists of C3 and C4 olefins.

In some embodiments, the first sulfuric acid solution has acid strength of at least about 96 percent, or at least about 97 percent, or at least about 98 percent. In some embodiments, the first sulfuric acid solution has acid strength in a range of from about 96 percent to about 99.5 percent, or from about 98.5 percent to about 99.2 percent. In some embodiments, the first sulfuric acid solution has acid strength of about 96.5 percent, or about 98.5 percent, or about 99.2 percent. In some embodiments, the first sulfuric acid solution is a fresh sulfuric acid solution.

In some embodiments, the second sulfuric acid solution comprises, consists essentially of, or consists of the first spent acid solution. In some embodiments, the second sulfuric acid solution further comprises an additional sulfuric acid solution having higher acid strength than the first spent acid solution. In some embodiments, sufficient amount of the additional sulfuric acid solution is added to the second reaction zone to maintain the acid strength of the second sulfuric acid solution in the range of from about 90 to about 99 percent. In some embodiments, the additional sulfuric acid solution is a fresh sulfuric acid solution.

The alkylate products produced in reaction zones can be combined and treated or purified. In some embodiments, the product mixtures or the effluents comprising spent acid solutions and alkylate products from reaction zones can be directed to an acid-hydrocarbon separation zone such as an acid settler wherein a hydrocarbon phase is separated from a sulfuric acid phase. The hydrocarbon phase exits the acid-hydrocarbon separation zone as a hydrocarbon effluent comprising alkylate, unreacted isoparaffin and residual sulfuric acid. In some embodiments, the hydrocarbon effluent passes through a tube bundle in an alkylation reactor and partially flashes to provide cooling to the alkylation reactor. In some embodiments, the hydrocarbon effluent can be directed to a liquid-vapor separation zone where the liquid-vapor separation is effected. In some embodiments, the hydrocarbon effluent can be purified to remove the residual sulfuric acid. In some embodiments, the hydrocarbon effluent can be directed to a fractionation separation zone to separate alkylate from the unreacted isoparaffin. All or part of the isoparaffin fraction generated in the fractionation separation zone can be recycled to the reaction zone.

In some embodiments, the alkylate product has a motor octane number (MON) of at least 90 or at least 92. In some embodiments, the alkylate product has a research octane number (RON) of at least 93 or at least 95. In some embodiments, the final boiling point of the alkylate product is in a range of from about 380° F. to about 440° F. In some embodiments, the final boiling point of the alkylate product is in a range of from about 390° F. to about 430° F. In some embodiments, the final boiling point of the alkylate product is in a range of from about 400° F. to about 420° F. In this disclosure, the final boiling point (FBP) of an alkylate product is determined using ASTM D86. All ASTM Standards are available from ASTM International, West Conshohocken, Pa., www.astm.org.

Many aspects and embodiments have been described above and are merely exemplary and not limiting. After reading this specification, skilled artisans appreciate that other aspects and embodiments are possible without departing from the scope of the invention.

### EXAMPLES

The concepts described herein will be further described in the following examples, which do not limit the scope of the invention described in the claims.

Examples 1-7 and 9 were carried out in three reaction zones disposed in sequence. Example 8 was carried out in four reaction zones disposed in sequence. The total amount of propylene fed to the first and second reaction zones was 5,660 barrels per day (BPD). The total amount of butylene fed to the first and second reaction zones was 2,115 barrels per day (BPD). Butylene used in the Examples 1-9 was a mixture of C4 olefins comprising 27 mole % isobutylene based on the total amount of butylene. It had an isomer distribution similar to the butylene from a refinery FCC (fluid catalytic cracking) unit. The isoparaffin reactant used in the Examples 1-9 was isobutane.

A diluent comprising propane and n-butane was also employed in the examples. The diluent ratio in each reaction zone was 0.2. The fresh sulfuric acid solution used in the Examples 1-9 had an acid strength of 99.2%. In each reaction zone, the volume ratio of the sulfuric acid solution to hydrocarbon was 1:1.

### Example 1

Three reaction zones were employed. In the first reaction zone, a C3 (propylene) and C4 (butylene) olefin mixture and isobutane were alkylated in the presence of a fresh sulfuric acid solution to produce a product mixture comprising a first spent acid solution and an alkylate product. The first spent acid solution was transferred and fed into the second reaction zone. In the second reaction zone, a C3 and C4 olefin mixture and isobutane were alkylated in the presence of the first spent acid solution to produce a product mixture comprising a second spent acid solution and an alkylate product. The second spent acid solution was transferred and fed into the third reaction zone.

In the third (final) reaction zone, butylene and isobutane were alkylated in the presence of the second spent acid solution to produce a product mixture comprising a final spent acid solution and an alkylate product. The amount of butylene fed to the final reaction zone was 5,473 barrels per day (BPD). In the final reaction zone, the olefin space velocity was 0.5 per hour, the reaction temperature was 45° F., and the molar ratio of isobutane to butylene fed to the reaction zone was 8. The final spent acid solution had an acid strength of 90.2%. The alkylate products from all three reaction zones were combined for analysis and yield calculation. Reaction conditions and results are shown in Table 1.

### Example 2

Same as Example 1 except as indicated in Table 1, and except that a fresh sulfuric acid solution was added as an additional sulfuric acid solution to the first spent acid solution prior to being fed into the second reaction zone, the amount of butylene fed to the final reaction zone was 19,350 barrels per day (BPD), and the final spent acid solution had an acid strength of 90%. Reaction conditions and results are shown in Table 1.

### Example 3

Same as Example 1 except as indicated in Table 1, and except that the final spent acid solution had an acid strength of 90%. Reaction conditions and results are shown in Table 1.

### Example 4

Same as Example 1 except as indicated in Table 1, and except that a fresh sulfuric acid solution was added as an additional sulfuric acid solution to the first spent acid solution prior to being fed into the second reaction zone, the amount of butylene fed to the final reaction zone was 17,250 barrels per day (BPD), and the final spent acid solution had an acid strength of 90%. Reaction conditions and results are shown in Table 1.

### Example 5

Same as Example 1 except as indicated in Table 1, and except that the final spent acid solution had an acid strength of 91.7%. Reaction conditions and results are shown in Table 1.

### Example 6

Same as Example 1 except as indicated in Table 1, and except that the final spent acid solution had an acid strength of 91.4%. Reaction conditions and results are shown in Table 1.

### Example 7

Same as Example 1 for the first and second reaction zones, except as indicated in Table 1.

In the third (final) reaction zone, C5 olefins (amylene) and isobutane were alkylated in the presence of the second spent acid solution to produce a product mixture comprising a final spent acid solution and an alkylate product. The amount of amylene fed to the final reaction zone was 6,152 barrels per day (BPD). In the final reaction zone, the olefin space velocity was 0.5 per hour, the reaction temperature was 45° F., and the molar ratio of isobutane to amylene fed to the reaction zone was 8. The final spent acid solution had an acid strength of 84%. The alkylate products from all three reaction zones were combined for analysis and yield calculation. Reaction conditions and results are shown in Table 1.

### Example 8

Example 8 was carried out in four reaction zones disposed in sequence. Same processes as Example 1 were carried out for the first and second reaction zones, except as indicated in Table 1.

In the third reaction zone, butylene and isobutane were alkylated in the presence of the second spent acid solution to produce a product mixture comprising a third spent acid solution and an alkylate product. The amount of butylene fed to the reactor was 5,473 barrels per day (BPD). In the third reaction zone, the olefin space velocity was 0.5 per hour, the reaction temperature was 45° F., and the molar ratio of isobutane to butylene fed to the reactor was 8. The third spent acid solution was transferred and fed into the fourth (final) reaction zone.

In the fourth (final) reaction zone, C5 olefins (amylene) and isobutane were alkylated in the presence of the third spent acid solution to produce a final spent acid solution and an alkylate product. The amount of amylene fed to the final reaction zone was 3,987 barrels per day (BPD). In the final reaction zone, the olefin space velocity was 0.5 per hour, the reaction temperature was 45° F., and the molar ratio of isobutane to amylene fed to the reactor was 8. The final spent acid solution has an acid strength of 84%. The alkylate products from all reaction zones were combined for analysis and yield calculation. Reaction conditions and results are shown in Table 1.

### Example 9 (Comparative)

Three reaction zones were employed. In the first reaction zone, a C3 (propylene) and C4 (butylene) olefin mixture and isobutane were alkylated in the presence of a fresh sulfuric acid solution to produce a first spent acid solution and an alkylate product. The first spent acid solution was transferred and fed into the second reaction zone. In the second reaction zone, butylene and isobutane were alkylated in the presence of the first spent acid solution to produce a second spent acid solution and an alkylate product. The second spent acid solution was transferred and fed into the third reaction zone.

In the third (final) reaction zone, butylene and isobutane were alkylated in the presence of the second spent acid solution to produce a final spent acid solution and an alkylate product. The amount of butylene fed to the final reaction zone was 5,473 barrels per day (BPD). In the final reaction zone, the olefin space velocity was 0.5 per hour, the reaction temperature was 45° F., and the molar ratio of isobutane to butylene fed to the reaction zone was 8. The final spent acid solution has an acid strength of 93.4%. The alkylate products from all three reaction zones were combined for analysis and yield calculation. Reaction conditions and results are shown in Table 1.

TABLE 1
Summary of Examples 1-9

| | First Reaction Zone | | | | Second Reaction Zone | | | | Acid | |
|---|---|---|---|---|---|---|---|---|---|---|
| Ex. No. | C3/C4 | T | SV | I/O | C3/C4 | T | SV | I/O | Cons. | Yield |
| 1 | 90/10 | 60 | 0.4 | 10 | 50/50 | 45 | 0.5 | 10 | 0.45 | 22,570 |
| 2 | 90/10 | 60 | 0.4 | 10 | 50/50 | 45 | 0.5 | 10 | 0.31 | 47,330 |
| 3 | 80/20 | 60 | 0.5 | 8 | 50/50 | 45 | 0.5 | 10 | 0.41 | 22,940 |
| 4 | 80/20 | 60 | 0.5 | 8 | 50/50 | 45 | 0.5 | 10 | 0.3 | 43,950 |
| 5 | 80/20 | 60 | 0.5 | 8 | 65/35 | 45 | 0.5 | 8 | 0.56 | 22,890 |
| 6 | 90/10 | 60 | 0.4 | 10 | 25/75 | 45 | 0.5 | 8 | 0.52 | 22,820 |
| 7 | 90/10 | 60 | 0.4 | 10 | 50/50 | 45 | 0.5 | 10 | 0.4 | 25,360 |
| 8 | 90/10 | 60 | 0.4 | 10 | 50/50 | 45 | 0.5 | 10 | 0.33 | 33,770 |
| 9 | 90/10 | 60 | 0.4 | 10 | 0/100 | 45 | 0.5 | 8 | 0.61 | 22,490 |

C3/C4 indicates the molar ratio of C3 to C4 olefins in the olefin mixture fed to the reaction zone; T indicates reaction temperature in Fahrenheit (° F.); SV is the olefin space velocity (per hour); I/O indicates the molar ratio of isobutane to olefin fed to the reaction zone; Acid Cons. means the acid consumption which is expressed in pound per gallon (lb/gal); Yield means the yield of the alkylate products which is expressed in barrels per day (BPD).

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination.

### EMBODIMENTS

For further illustration, additional non-limiting embodiments of the present disclosure are set forth below.

For example, embodiment 1 is an alkylation process comprising: (a) providing three or more reaction zones disposed in sequence; (b) contacting a non-final olefin mixture comprising C3 and C4 olefins with an isoparaffin comprising isobutane in the presence of a non-final sulfuric acid solution under effective alkylation conditions in each non-final reaction zone to produce a product mixture comprising a non-final spent acid solution and an alkylate product; and (c) contacting a final olefin comprising an olefin selected from the group consisting of C4 olefins, C5 olefins and mixtures thereof with an isoparaffin comprising isobutane in the presence of a final sulfuric acid solution under effective alkylation conditions in a final reaction zone to produce a product mixture comprising a final spent acid solution and an alkylate product; wherein the molar ratio of C3 to C4 olefins in the non-final olefin mixture decreases in each subsequent non-final reaction zone, and the sulfuric acid solution present in a reaction zone comprises the spent acid solution produced in the immediately preceding reaction zone.

Embodiment 2 is an alkylation process as set forth in embodiment 1 wherein the sulfuric acid solution present in a reaction zone between a first reaction zone and the final reaction zone further comprises an additional sulfuric acid solution having higher acid strength than the spent acid solution produced in the immediately preceding reaction zone.

Embodiment 3 is an alkylation process as set forth in embodiment 2 wherein the sulfuric acid solution present in a second reaction zone further comprises an additional sulfuric acid solution having higher acid strength than the spent acid solution produced in the first reaction zone.

Embodiment 4 is an alkylation process as set forth in embodiments 2 or 3 wherein the additional sulfuric acid solution is a fresh sulfuric acid solution.

Embodiment 5 is an alkylation process as set forth in any of the preceding embodiments wherein the non-final olefin mixture in step (b) is essentially free of C5 olefins.

Embodiment 6 is an alkylation process as set forth in any of the preceding embodiments wherein the molar ratio of C3 to C4 olefins in the non-final olefin mixture in step (b) is in a range of from about 95:5 to about 30:70.

Embodiment 7 is an alkylation process as set forth in any of the preceding embodiments wherein the isoparaffin comprises at least about 80 mole % of isobutane based on the total amount of the isoparaffin.

Embodiment 8 is an alkylation process as set forth in any of the preceding embodiments wherein the final sulfuric acid solution in the final reaction zone is essentially free of additional sulfuric acid solution.

Embodiment 9 is an alkylation process as set forth in any of the preceding embodiments wherein the final olefin comprises C4 olefin(s) essentially free of C3 and C5 olefins, and the final spent acid solution has acid strength in a range of from about 87 percent to about 91 percent.

Embodiment 10 is an alkylation process as set forth in one of embodiments 1-8, wherein the final olefin comprises C5 olefin(s) essentially free of C3 and C4 olefins, and the final spent acid solution has acid strength in a range of from about 80 percent to about 85 percent.

Embodiment 11 is an alkylation process as set forth in any of the preceding embodiments wherein acid consumption is no more than about 0.7 lb/gal.

Embodiment 12 is an alkylation process comprising: (a) providing three reaction zones disposed in sequence; (b) contacting a first olefin mixture comprising C3 and C4 olefins with an isoparaffin comprising isobutane in the presence of a first sulfuric acid solution under effective alkylation conditions in a first reaction zone to produce a product mixture comprising a first spent acid solution and an alkylate product; (c) contacting a second olefin mixture comprising C3 and C4 olefins with an isoparaffin comprising isobutane in the presence of a second sulfuric acid solution comprising the first spent acid solution under effective alkylation conditions in a second reaction zone to produce a product mixture comprising a second spent acid solution and an alkylate product; and (d) contacting a final olefin comprising an olefin selected from the group consisting of C4 olefins, C5 olefins and mixtures thereof with an isoparaffin comprising isobutane in the presence of a final sulfuric acid solution comprising the second spent acid solution under effective alkylation conditions in a final reaction zone to produce a product mixture comprising a final spent acid solution and an alkylate product; wherein the molar ratio of C3 to C4 olefins in the first olefin mixture is in a range of from about 99:1 to about 60:40, the molar ratio of C3 to C4 olefins in the second olefin mixture is in a range of from about 70:30 to about 30:70, and the molar ratio of C3 to C4 olefins in the first olefin mixture is higher than the molar ratio of C3 to C4 olefins in the second olefin mixture.

Embodiment 13 is an alkylation process comprising: (a) providing four reaction zones disposed in sequence; (b) contacting a first olefin mixture comprising C3 and C4 olefins with an isoparaffin comprising isobutane in the presence of a first sulfuric acid solution under effective alkylation conditions in a first reaction zone to produce a product mixture comprising a first spent acid solution and an alkylate product; (c) contacting a second olefin mixture comprising C3 and C4 olefins with an isoparaffin comprising isobutane in the presence of a second sulfuric acid solution comprising the first spent acid solution under effective alkylation conditions in a second reaction zone to produce a product mixture comprising a second spent acid solution and an alkylate product; (d) contacting a third olefin comprising a C4 olefin with an isoparaffin comprising isobutane in the presence of a third sulfuric acid solution comprising the second spent acid solution under effective alkylation conditions in a third reaction zone to produce a product mixture comprising a third spent acid solution and an alkylate product; and (e) contacting a final olefin comprising a C5 olefin with an isoparaffin comprising isobutane in the presence of a final sulfuric acid solution comprising the third spent acid solution under effective alkylation conditions in a final reaction zone to produce a product mixture comprising a final spent acid solution and an alkylate product; wherein the molar ratio of C3 to C4 olefins in the first olefin mixture is in a range of from about 99:1 to about 60:40, the molar ratio of C3 to C4 olefins in the second olefin mixture is in a range of from about 70:30 to about 30:70, and the molar ratio of C3 to C4 olefins in the first olefin mixture is higher than the molar ratio of C3 to C4 olefins in the second olefin mixture.

Embodiment 14 is an alkylation process as set forth in embodiment 13 wherein the third olefin consists essentially of a C4 olefin.

Embodiment 15 is an alkylation process as set forth in embodiments 13 or 14 wherein the final olefin consists essentially of a C5 olefin.

Embodiment 16 is an alkylation process as set forth in one of embodiments 13-15, wherein essentially no fresh sulfuric acid solution is fed into the third reaction zone.

Embodiment 17 is an alkylation process as set forth in one of embodiments 12-16, wherein the molar ratio of C3 olefin to C4 olefins in the first olefin mixture is in a range of from about 90:10 to about 70:30.

Embodiment 18 is an alkylation process as set forth in one of embodiments 12-17, wherein the first olefin mixture is essentially free of C5 olefins.

Embodiment 19 is an alkylation process as set forth in one of embodiments 12-18, wherein the second olefin mixture is essentially free of C5 olefins.

Embodiment 20 is an alkylation process as set forth in one of embodiments 12-19, wherein the second sulfuric acid solution further comprises an additional sulfuric acid solution having higher acid strength than the first spent acid solution.

Embodiment 21 is an alkylation process as set forth in embodiment 20 wherein the additional sulfuric acid solution is a fresh sulfuric acid solution.

Embodiment 22 is an alkylation process as set forth in one of embodiments 12-21, wherein essentially no fresh sulfuric acid solution is fed into the final reaction zone.

Embodiment 23 is an alkylation process comprising: (a) providing two reaction zones disposed in sequence; (b) contacting a first olefin mixture comprising C3 and C4 olefins with an isoparaffin comprising isobutane in the presence of a first sulfuric acid solution under effective alkylation conditions in a first reaction zone to produce a product mixture comprising a first spent acid solution and an alkylate product; and (c) contacting a second olefin mixture comprising C3 and C4 olefins with an isoparaffin comprising isobutane in the presence of a second sulfuric acid solution comprising the first spent acid solution under effective alkylation conditions in a second reaction zone to produce a product mixture comprising a second spent acid solution and an alkylate product; wherein the molar ratio of C3 olefin to C4 olefins in the first olefin mixture is in a range of from about 99:1 to about 60:40, the molar ratio of C3 olefin to C4 olefins in the second olefin mixture is in a range of from about 70:30 to about 30:70, and the molar ratio of C3 olefin to C4 olefins in the first olefin mixture is higher than the molar ratio of C3 olefin to C4 olefins in the second olefin mixture.

What is claimed is:

1. An alkylation process comprising:
(a) providing three or more reaction zones disposed in sequence;
(b) contacting a non-final olefin mixture comprising C3 and C4 olefins with an isoparaffin comprising isobutane in the presence of a non-final sulfuric acid solution under effective alkylation conditions in each non-final reaction zone to produce a product mixture comprising a non-final spent acid solution and an alkylate product; and
(c) contacting a final olefin comprising an olefin selected from the group consisting of C4 olefins, C5 olefins and mixtures thereof with an isoparaffin comprising isobutane in the presence of a final sulfuric acid solution under effective alkylation conditions in a final reaction zone to produce a product mixture comprising a final spent acid solution and an alkylate product;
wherein the molar ratio of C3 to C4 olefins in the non-final olefin mixture decreases in each subsequent non-final reaction zone, and the sulfuric acid solution present in a reaction zone comprises the spent acid solution produced in the immediately preceding reaction zone.

2. The alkylation process of claim 1 wherein the sulfuric acid solution present in a reaction zone between a first reaction zone and the final reaction zone further comprises an additional sulfuric acid solution having higher acid strength than the spent acid solution produced in the immediately preceding reaction zone.

3. The alkylation process of claim 1 or 2 wherein the non-final olefin mixture in step (b) is essentially free of C5 olefins.

4. The alkylation process as in any of the preceding claims wherein the molar ratio of C3 to C4 olefins in the non-final olefin mixture in step (b) is in a range of from about 95:5 to about 30:70.

5. The alkylation process as in any of the preceding claims wherein the final sulfuric acid solution in the final reaction zone is essentially free of additional sulfuric acid solution.

6. The alkylation process as in any of the preceding claims wherein acid consumption is no more than about 0.7 lb/gal.

7. An alkylation process comprising:
(a) providing three reaction zones disposed in sequence;
(b) contacting a first olefin mixture comprising C3 and C4 olefins with an isoparaffin comprising isobutane in the presence of a first sulfuric acid solution under effective alkylation conditions in a first reaction zone to produce a product mixture comprising a first spent acid solution and an alkylate product;
(c) contacting a second olefin mixture comprising C3 and C4 olefins with an isoparaffin comprising isobutane in the presence of a second sulfuric acid solution comprising the first spent acid solution under effective alkylation conditions in a second reaction zone to produce a product mixture comprising a second spent acid solution and an alkylate product; and
(d) contacting a final olefin comprising an olefin selected from the group consisting of C4 olefins, C5 olefins and mixtures thereof with an isoparaffin comprising isobutane in the presence of a final sulfuric acid solution comprising the second spent acid solution under effective alkylation conditions in a final reaction zone to produce a product mixture comprising a final spent acid solution and an alkylate product;
wherein the molar ratio of C3 to C4 olefins in the first olefin mixture is in a range of from about 99:1 to about 60:40, the molar ratio of C3 to C4 olefins in the second olefin mixture is in a range of from about 70:30 to about 30:70, and the molar ratio of C3 to C4 olefins in the first olefin mixture is higher than the molar ratio of C3 to C4 olefins in the second olefin mixture.

8. An alkylation process comprising:
(a) providing four reaction zones disposed in sequence;
(b) contacting a first olefin mixture comprising C3 and C4 olefins with an isoparaffin comprising isobutane in the presence of a first sulfuric acid solution under effective alkylation conditions in a first reaction zone to produce a product mixture comprising a first spent acid solution and an alkylate product;
(c) contacting a second olefin mixture comprising C3 and C4 olefins with an isoparaffin comprising isobutane in the presence of a second sulfuric acid solution comprising the first spent acid solution under effective alkylation conditions in a second reaction zone to produce a product mixture comprising a second spent acid solution and an alkylate product;
(d) contacting a third olefin comprising a C4 olefin with an isoparaffin comprising isobutane in the presence of a third sulfuric acid solution comprising the second spent acid solution under effective alkylation conditions in a third reaction zone to produce a product mixture comprising a third spent acid solution and an alkylate product; and
(e) contacting a final olefin comprising a C5 olefin with an isoparaffin comprising isobutane in the presence of a final sulfuric acid solution comprising the third spent acid solution under effective alkylation conditions in a final reaction zone to produce a product mixture comprising a final spent acid solution and an alkylate product;
wherein the molar ratio of C3 to C4 olefins in the first olefin mixture is in a range of from about 99:1 to about 60:40, the molar ratio of C3 to C4 olefins in the second olefin mixture is in a range of from about 70:30 to about 30:70, and the molar ratio of C3 to C4 olefins in the first olefin mixture is higher than the molar ratio of C3 to C4 olefins in the second olefin mixture.

9. The alkylation process of claim 7 or 8, wherein the molar ratio of C3 olefin to C4 olefins in the first olefin mixture is in a range of from about 90:10 to about 70:30.

10. An alkylation process comprising:
(a) providing two reaction zones disposed in sequence;
(b) contacting a first olefin mixture comprising C3 and C4 olefins with an isoparaffin comprising isobutane in the presence of a first sulfuric acid solution under effective alkylation conditions in a first reaction zone to produce a product mixture comprising a first spent acid solution and an alkylate product; and
(c) contacting a second olefin mixture comprising C3 and C4 olefins with an isoparaffin comprising isobutane in the presence of a second sulfuric acid solution comprising the first spent acid solution under effective alkylation conditions in a second reaction zone to produce a product mixture comprising a second spent acid solution and an alkylate product;
wherein the molar ratio of C3 olefin to C4 olefins in the first olefin mixture is in a range of from about 99:1 to about 60:40, the molar ratio of C3 olefin to C4 olefins in the second olefin mixture is in a range of from about 70:30 to about 30:70, and the molar ratio of C3 olefin to C4 olefins in the first olefin mixture is higher than the molar ratio of C3 olefin to C4 olefins in the second olefin mixture.
